(19) United States
(12) Patent Application Publication
(10) Pub. No.: US 2005/0004155 A1
(43) Pub. Date: Jan. 6, 2005

(54) Use of methylnaltrexone to treat irritable bowel syndrome

(76) Inventors: Thomas A. Boyd, Grandview, NY (US); Robert J. Israel, Suffern, NY (US); Suketu P. Sanghvi, Kendall Park, NJ (US)

Correspondence Address: Edward R. Gates;Wolf, Greenfield & Sacks, P.C., 600 Atlantic Avenue, Boston, MA, 02210, US

(21) Appl. No.: 10/821,813

(22) Filed: Apr. 8, 2004

Related U.S. Application Data
(60) Provisional application No. 60/461,608, filed on Apr. 8, 2003.

(51) Int. Cl. A61K 31/485

(52) U.S. Cl. 514/282; 514/279

(57) ABSTRACT

Methods of treating irritable bowel syndrome with peripheral opioid antagonists, such as methylnaltrexone, are provided. Formulations comprising peripheral opioid antagonists, such as methylnaltrexone, and irritable bowel syndrome therapeutic agents are also provided.

### FIELD OF THE INVENTION

The invention relates to the field of treating irritable bowel syndrome. In particular, the invention relates to the discovery that irritable bowel syndrome is treatable by administration of peripheral opioid antagonists such as methylnaltrexone.

### BACKGROUND OF THE INVENTION

Irritable bowel syndrome (IBS) is a gastrointestinal disorder characterized by altered bowel habits and abdominal pain, typically in the absence of detectable structural abnormalities. IBS is one of the most common conditions but one of the least well understood in clinical practice. The definition of IBS is based on its clinical presentation, since no clear diagnostic markers exist for IBS. IBS is often confused with inflammatory bowel disease (IBD), colitis, mucous colitis, spastic colon, or spastic bowel. The Rome criteria can be used to diagnose lBS and rule out other disorders. The Rome criteria include abdominal pain and/or discomfort which is relieved with defecation and/or a change in stool frequency and/or a change in stool consistency for at least three months and two or more of a change in stool frequency, change in consistency, difficult stool passage, sense of incomplete evacuation, and presence of mucous in stool, at least 25% of the time for at least three months (see, Harrison's Principals of Internal Medicine; Braunwald, E., et al. Ed.; McGraw-Hill: New York 2001, hereby incorporated by reference). Only recently have physicians generally considered IBS to be a disease, rather than a somatic manifestation of psychological stress. Although progress has been made towards a better understanding of the pathogenesis of IBS, improved methods of treatment are necessary as no satisfactory treatments are currently available.

IBS is present in approximately 20% of the adult population in the United States. IBS is common in a young population, with most new cases presenting before age 45. However, some elderly patients are troubled by the symptoms of IBS, as are children. Women are diagnosed with IBS two to three times as often as men and make up 80% of the population diagnosed with severe IBS. Although IBS is not life-threatening, it is painful and can be socially debilitating.

IBS patients typically fall into two broad clinical groups. Most IBS patients fall into the first group, which have abdominal pain associated with altered bowel habits that include constipation, diarrhea, or alternating constipation and diarrhea. The second group of IBS patients comprises patients with painless diarrhea. Most IBS patients experience several IBS symptoms such as abdominal pain, altered bowel habits, gas, flatulence, upper gastrointestinal symptoms, e.g., dyspepsia, heartburn, nausea, vomiting. Many patients also suffer from depression as an indirect result of IBS.

The pathogenesis of IBS is poorly understood; it has been proposed that abnormal gut motor and sensory activity, central neural dysfunction, psychological disturbances, stress, and luminal factors each play a role.

It is generally believed that the central nervous system role is important in the pathogenesis of IBS. This role is strongly suggested by the clinical association of emotional disorders with IBS symptom exacerbation, the clinical association of stress with IBS symptom exacerbation, and the therapeutic response to IBS therapies that act on cerebral cortical sites. Additionally, positron omission tomography has shown alterations in regional cerebral blood flow in IBS patients relative to healthy individuals. For example, in healthy individuals, rectal distention increases blood flow in the anterior cingulate cortex, a region with an abundance of opiate receptors. When activated, these central opiate receptors may help to reduce sensory input. However, IBS patients do not exhibit increased blood flow in the anterior cingulate cortex, but show activation of the prefrontal cortex in response to rectal activation or in response to anticipation of rectal distension. Activation of the frontal lobes is thought to activate a vigilance network within the brain that increases alertness. The anterior cingulate cortex and the prefrontal cortex are believed to have reciprocal inhibitory associations. In IBS patients, the preferential activation of the prefrontal lobe without activation of the anterior cingulate cortex is believed to be a form of cerebral dysfunction leading to the increased perception of visceral pain. Patients with IBS frequently demonstrate increased motor reactivity of the colon and small bowel to a variety of stimuli and altered visceral sensation associated with lower sensation thresholds, which are believed to result from central nervous system dysregulation.

Alterations in gut motility have been detected in IBS. For example, patients with constipation-predominant IBS have fewer propulsive contractions after eating (Talley, N. J., and Spiller, R., Lancet 2002;360:555-564). Those with diarrhea-predominant IBS may have shorter small bowel and colonic transit times than those with constipation. The altered motor response in gut tissue in patients with IBS may be due partly to exaggerated responses to stimuli related to brain-gut dysregulation. It is unknown whether alterations localized in the gut region play a significant role.

Opioids may be involved in the control of gut motility. Exogenous opioids such as morphine inhibit intestinal propulsion by mechanisms that include both central and peripheral components (Manara, L., and Bianchetti, A., Ann. Rev. Pharmacol. Toxicol. 1985;25:249-273). It is well known that the administration of exogenous opioids for the purpose of inducing analgesia in patients who are suffering from pain will often result in gastrointestinal side effects such as gastric and bowel hypomotility, which in turn contribute to poor digestion, constipation, and discomfort. A direct action of opioids on the gut has been established. For example, endogenous opioids are found in the intestine. These include the opioid peptides encephalin, dynorphin, and endorphin. The endogenous opioid peptides induce segmentation and inhibit peristalsis in the intestine (Kromer, W., Dig. Dis. 1990;8:361-373). Further, opioids in the gut have the potential to increase smooth muscle tone, alter electrolyte absorption, and change the secretory functions of the gut wall. In the intestine, endogenous opioids reside in the enteric nervous system, a system of neurons located between the layers of circular smooth muscle and longitudinal smooth muscle in the gut wall, and which are especially concentrated in the myenteric plexus and the submucosal plexus. Mu, kappa, and delta opioid receptors have been identified in these cells (Hedner, T., and Cassuto, J., Scand. J. Gastroenterol. Suppl. 1987;130:27-46). Endogenous and exogenous opioids appear to act principally by binding to opioid receptors on acetylcholine-containing nerves in the gut, hyperpolarizing the cells, and inhibiting the release of acetylcholine from presynaptic nerve terminals. The reduced acetylcholine release may be the immediate effector mechanism by which bowel function is slowed or otherwise disrupted from its normal segmentation/propulsion sequences. The side effects involving bowel hypomotility that accompany the use of exogenous opioids for analgesia might be exaggerated responses to normal opioid functions in this organ.

Treatment of IBS with centrally acting opioid antagonists has not been successfully demonstrated. The centrally available opioid antagonist naloxone has been tested in small trials without success. Hawkes, et al. conducted a randomized, double-blind, placebo-controlled trial in 25 subjects who fulfilled the Rome criteria for IBS and who exhibited IBS of the constipation-predominant and alternating types (Hawkes, N. D., et al., Aliment. Pharmacol. Ther. 2002;16:1649-1654). Subjects were administered a treatment regimen consisting of placebo or of 1 mg naloxone twice daily for 8 weeks. When the principal endpoint of “adequate symptom relief” was examined, the results in the naloxone-treated group were not statistically significantly different from those in the placebo-treated group. Marginal but non-statistically significant improvements in subjective ratings such as severity ratings and pain scores were noted; however, the interpretation of these findings with respect to a specific gastrointestinal effect of the opioid antagonist is complicated by the possibility that naloxone also enters the central nervous system. In a separate study, naloxone 0.4 mg or placebo was administered intravenously to 50 consecutive patients to present at hospital with IBS. The degree of muscle spasm and the relative intensity of pain was determined by means of air insufflation during sigmoidoscopy. Treatment with naloxone was not associated with any objective or subjective evidence of beneficial effect (Fielding, J. F., and O'Malley, K., Ir. J. Med. Sci., 1981;150:41-2).

In another study, a derivative of the opioid receptor antagonist nalmefene, namely nalmefene glucuronide, was studied in eight patients with constipation-predominant IBS (Chalmi, T. N., et al., Am. J. Gastroenterol. 1993;88:1568 [abstract]). Over an eight week period, patients were administered 16 mg nalmefene glucuronide three times a week. Patients reported decreased gut transit time and increased stool frequency; however, the compound did not reduce abdominal pain or bloating, and stool consistency was not improved.

U.S. Pat. No. 6,395,705 describes the use of “excitatory” opioid antagonists to treat IBS. The '705 patent teaches using extremely low doses of such antagonists, lower than doses used conventionally to counteract the side-effects of opioid treatment (such as gut hypomotility). The “excitatory” antagonists listed are centrally acting and act on both central and peripheral opioid receptors.

Throughout the body, it is believed that calcium channels of cells within the central nervous system are involved in the pathogenesis of endorphin-mediated pathologies such as IBS. These pathologies are characterized by elevated, free and bound endorphin levels, as described in U.S. Pat. No. 5,811,451, hereby incorporated by reference. U.S. Pat. No. 5,811,451 posits that these increased tissue and circulating levels of endorphins affect calcium metabolism. When endorphins increased beyond certain physiological limits, cellular calcium ion flow is impaired, resulting in “endocellular and endotissutal” calcium deficits with an increase of calcemia. As a result, it was believed that increased endocellular calcium request signaling caused recruitment of external calcium towards the damaged tissues, thereby causing endorphins to accumulate. Although the presence of bound endorphins to nervous system receptors is normal at certain levels, the increase in bound endorphins caused by the calcium deficit causes a large amount of neuromodulators to accumulates forming an “endorphin cloud.” The endorphin cloud alters the membrane potential and permeability in the nervous system cells as well as other cells having endorphin receptors. The alteration of the cell permeability caused by the calcium deficit influences the activity and functionality of calcium channels and the related consequent activities and functions. Calcium has been administered in conjunction with opiate antagonists to prevent calcium outflow from cells, thereby preventing worsening of the cellular damage and treating endorphin-mediated pathologies such as IBS.

Opioid antagonists in combination with calcium salts have been described in U.S. Pat. No. 5,811,451. The administration of calcium in conjunction with the opioid antagonists was thought to be critical to prevent further calcium outflow from cells into the bloodstream, as the cells were already impaired by calcium ion deficit.

Although the administration of calcium is beneficial in the treatment of endorphin-mediated pathologies such as IBS, it is often not desirable to administer calcium, for example, as many people suffer from hypercalcemia, an excessive amount of calcium in the blood.

Parathyroid hormone (PTH) and vitamin D regulate calcium balance in the body. Elevated levels of PTH, often caused by primary hyperparathyroidism, is the most common cause of hypercalcemia. Elevated PTH levels also cause hypercalcemia found in patients with familial hypocalciuric hypercalcemia. Many cancer patients with hypercalcemia have normal levels of PTH, as malignant tumors often produce PTH-related protein (PTHrP) which also raises blood calcium levels.

Another common cause of hypercalcemia is excess of vitamin D, as a result of diet or disorders such as granulomatous diseases. Hypercalcemia can also result from kidney failure, adrenal gland failure, hyperthyroidism, prolonged immobilization, use of therapeutic agents such as thiazides, and ingestion or administration of large amounts of calcium.

There are a variety of symptoms of hypercalcemia, including abdominal symptoms, skeletal symptoms such as bone pain, kidney symptoms such as flank pain and kidney stones, psychological symptoms such as depression and irritability, and muscular symptoms such as muscle atrophy.

The abdominal symptoms of hypercalcemia include abdominal pain, nausea, vomiting, poor appetite, and constipation. Since IBS patients typically also suffer from these symptoms, it is undesirable to administer exogenous calcium to these patients, since calcium could potentially exacerbate their symptoms.

### SUMMARY OF THE INVENTION

One of the underlying pathophysiological causes contributing to altered gut motility in irritable bowel syndrome may be an interruption of normal peristalsis with a resultant predominance of segmentation. Without normal peristalsis, the movement of gut contents slows or ceases. These might be contributory factors to the clinical symptoms of constipation and pain, for example, in patients with irritable bowel syndrome of the constipation or constipation/pain spectrum of symptoms. Given that endogenous opioids are possible mediators in the control of gut segmentation and peristalsis which are disturbed in IBS, applicants believe that a peripherally acting opioid antagonist such as methylnaltrexone would be beneficial in the treatment of irritable bowel syndrome.

The invention is based, in part, on the surprising discovery that the administration of peripheral opioid antagonists such as quaternary derivatives of noroxymorphone in the absence of calcium can be used to treat irritable bowel syndrome (IBS). Because of the uncertainty in the mechanism of irritable bowel syndrome, the strong evidence of a central nervous system role, and the known importance of administrating calcium ions to treat endorphin-mediated pathologies such as IBS, it was unpredictable and unexpected that peripheral opioid antagonists such as quaternary derivatives of noroxymorphone, which do not have central nervous system effects, in the absence of calcium are effective therapeutic agents for treating irritable bowel syndrome.

In one aspect of the invention, methods for treating irritable bowel syndrome are provided. The methods comprise administering to patients in need of such treatment an effective amount of a pharmaceutical preparation comprising a peripheral opioid antagonist and free of bioavailable calcium and salts thereof to ameliorate at least one symptom of the irritable bowel syndrome. In some embodiments, the pharmaceutical preparations are administered parenterally. In other embodiments, the pharmaceutical preparations are administered intravenously, subcutaneously, intramuscularly, via needless injection, and via infusion. In other embodiments, the pharmaceutical preparation is administered intrarectally, intranasally and transdermally. In some embodiments, the pharmaceutical preparation is formulated as a solution. In other embodiments the pharmaceutical preparation is formulated as a suppository. In other embodiments the pharmaceutical preparation is formulated as an enema, tablet, capsule, or transdermal formulation. The preferred peripheral opioid antagonists are mu opioid antagonists such as quaternary derivatives of noroxymorphone, piperdine-N-alkylcarboxylates, opium alkaloid derivatives, and quaternary benzomorphans. The most preferred antagonist is methylnaltrexone, a quaternary derivative of noroxymorphone.

In another aspect of the invention, methods are provided for treating IBS, by orally administering a pharmaceutical preparation comprising a peripheral opioid antagonist and free of bioavailable calcium and salts thereof, is administered to a patient in need of such treatment in an effective amount. Important embodiments including preferred opioid antagonists are as described above.

The IBS symptoms that may be ameliorated by the methods of the invention include abdominal pain, abdominal distension, abnormal stool consistency, abnormal stool frequency, altered bowel habits, bloating (e.g., abdominal bloating), constipation, diarrhea, alternating diarrhea and constipation, flatulence, gas, mucous in the stool, and upper gastrointestinal symptoms including dyspepsia, heartburn, nausea and vomiting. In some embodiments, one symptom is ameliorated. In other embodiments, two or more symptoms are ameliorated. The symptoms ameliorated may be any one, any combination of two or more, or all of the foregoing symptoms. Each such combination is intended to be included as if specifically recited herein.

In some embodiments of the invention, the patients are also administered antibiotics. In some embodiments of the invention, the patients are also administered an irritable bowel syndrome therapeutic agent. Irritable bowel syndrome therapeutic agents that may be administered to the patient to ameliorate at least one symptom of IBS include antispasmodics, anti-muscarinics, antidiarrheals, antiinflammatory agents, pro-motility agents, 5HT₁ agonists, 5HT₃ antagonists, 5HT₄ antagonists, 5HT₄ agonists, bile salt sequestering agents, bulk-forming agents, bulk-forming laxatives, cathartic laxatives, diphenylmethane laxatives, osmotic laxatives, saline laxatives, other laxatives, stool softeners, alpha2-adrenergic agonists, mineral oils, antidepressants, and herbal medicines.

A preferred quaternary derivative of noroxymorphone for all of the methods and formulations described herein is methylnaltrexone and salts thereof.

The peripheral opioid antagonist may be administered using any commercial mode of administration or any mode of administration known to those of skill in the art. The opioid antagonist may be administered enterally or parenterally. These modes of administration include, but are not limited to, intravenous, subcutaneous, oral, transdermal, transmucosal, topical, and rectal administration. Additionally, the peripheral opioid antagonist may be administered as an enterically coated tablet or capsule. In some embodiments, the opioid antagonist is administered by an infusion method (e.g., a slow infusion method) or by a time-release method. In other embodiments, the opioid antagonist is administered as a suppository or enema.

In any of the aspects and embodiments of the invention described above, the peripheral opioid antagonist typically is administered in an amount ranging from 0.01 to 1000 mg per day.

When the peripheral opioid antagonist is administered parenterally, such as intravenously or subcutaneously, the dosage typically may range from 0.001 to 5.0 mg/kg body weight of the patient. In some embodiments, the dosage may range from 0.001 to 0.45 mg/kg body weight of the patient. In other embodiments, the dosage may range from 0.1 to 0.3 mg/kg body weight of the patient. For subcutaneous administration, it is preferred to administer a volume of 0.5 to 1.5 cc to the patient to avoid pain.

In some embodiments, the peripheral opioid antagonist is administered orally in an amount ranging from 10 to 750 mg/day. In other embodiments, the amount ranges from 50 to 250 mg/day. In a particular embodiment, the amount is 75 mg. In another particular embodiment, the amount is 225 mg. The dosage depends on the formulation used, for example, oral doses with enteric coatings are typically administered in amounts lower than oral doses that are not enterically coated. Suitable dosage units can be readily determined by those of skill in the art.

In some embodiments, the methods of the invention described herein results in mean peak plasma concentrations of 1400 mg/ml or less of peripheral opioid antagonist. In some embodiments, the mean peak plasma concentration is 1200 mg/ml or less. In other embodiments, the mean peak concentration is 1000 mg/ml or less.

In some embodiments of the invention, the patient's plasma level of the peripheral opioid antagonist does not exceed 1000 ng/ml. The peripheral opioid antagonist may be administered in an effective amount such that the patient's mean peak plasma level of the quaternary derivative does not exceed 2000, 1500, 750, 500, 400, 300, 250, 200, 150, 100, 50, or even 20 ng/ml. In other embodiments, the peripheral opioid antagonist is administered in an amount to maintain the patient's mean peak plasma levels of 1400 ng/ml or less; 1200 ng/ml or less; 1000, 500, 400, 300, 200, 100, or even 20 ng/ml. Patient drug plasma levels may be measured using routine HPLC methods known to those of skill in the art.

In some embodiments of the invention, the pharmaceutical preparation is orally administered in an enteric coated formulation. In other embodiments, the pharmaceutical preparation is administered as a slow release formulation. In a further embodiment, the pharmaceutical preparation is administered as an enteric-coated, sustained release formulation. In still other embodiments, the quaternary derivative is administered in a colonic site-directed formulation.

In some embodiments, the patients treatable by the methods of the invention are adults. In other embodiments, the patients are children. In some embodiments of the invention, the patients treatable by the methods of the invention are female. In other embodiments, the patients are male. In some embodiments, the patients are younger than 60, and in other embodiments, the patients are over 60 years old.

In some embodiments of the invention, the peripheral opioid antagonist is administered to the patient in an amount effective to ameliorate at least one symptom of IBS. In other embodiments, two or more symptoms are ameliorated.

In some embodiments of the invention, the patients are not administered exogenous opioids, i.e., not undergoing exogenous opioid treatment. In other embodiments, the patients are administered exogenous opioids, for example, as therapy for pain, i.e., undergoing opioid treatment. In some of these embodiments, the patients are administered opioid chronically, that is, for one week or more. In some embodiments, the opioid is alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, opium, oxycodone, oxymorphone, pentazocine, propiram, propoxyphene, remifentanyl, sufentanil, tilidine, trimebutine, and tramadol. In a particular embodiment, the opioid is loperamide. In other embodiments, the opioid is a mixed agonist such as butorphanol. In some embodiments, the patients are administered more than one opioid, for example, morphine and heroin or methadone and heroin.

In another aspect of the invention, compositions comprising a peripheral opioid antagonist and an irritable bowel syndrome therapeutic agent are provided. In yet another aspect of the invention, compositions comprising a peripheral opioid antagonist and an antibiotic are provided. Preferred peripheral opioid antagonists are as described above. The compositions described above may additionally comprise an opioid agonist. The compositions may further comprise a pharmaceutically acceptable carrier and be pharmaceutical preparations.

In some embodiments, the pharmaceutical preparation are formulated for oral administration. Formulations for oral administration include a capsule (e.g., a solid-filled capsule), a powder, a granule, a crystal, a tablet, a solution, an extract, a suspension, a soup, a syrup, an elixir, a tea, a liquid-filled capsule, an oil, a chewable tablet, a chewable piece, an enteric-coated tablet, sustained-release, a site-specific release dosage form, and an enteric-coated sustained release tablet or capsule.

In some embodiments, the pharmaceutical preparation is formulated for rectal administration. Formulations for rectal administration include suspensions, solutions, suppositories, oils, and enemas.

In other embodiments, the pharmaceutical preparation is formulated for sublingual, intranasal, transdermal, intradermal, intramuscular, subcutaneous, injectable, and infusion administration.

According to another aspect of the invention, kits are provided. The kit is a package containing a preparation of a peripheral opioid antagonist and a preparation of an antibiotic and/or an IBS therapeutic agent. The kit can optionally contain instructions for administering the antagonist and the antibiotic and/or IBS therapeutic agent to a subject. The peripheral opioid antagonist and the antibiotic and/or IBS therapeutic agent may be in the same or different formulation. The kit may include any of the formulations described above or throughout the specification. The kit may also include an administration device for administering one or more of the preparations. The administration device can be any means useful in administering one of the preparations in the kit, such as a syringe, an enema, a glove, an infusion set, an inhaler, a spray device, a tube, etc.

According to another aspect of the invention, a method of manufacture is provided. The method involves combining a peripheral opioid antagonist with an antibiotic and/or IBS therapeutic agent to provide a formulation according to the invention. The method can further comprise combining a pharmaceutically acceptable carrier and/or an opioid and the antibiotic, and/or therapeutic agent with the antagonist to provide the formulation. The antagonist antibiotic and/or IBS therapeutic agent (and optionally opioid) and carrier.

### BRIEF DESCRIPTION OF THE DRAWING

FIG. 1 illustrates a kit according to the invention.

### DETAILED DESCRIPTION

The present invention provides methods for treating irritable bowel syndrome (IBS) comprising administering an effective amount of a peripheral opioid antagonist to ameliorate at least one symptom of IBS.

Peripheral opioid antagonists are well-known in the art. Peripheral opioid antagonists, as used herein, means those opioid antagonists which do not effectively cross the blood-brain barrier into the central nervous system. The majority of currently known opioid antagonists act both centrally and peripherally, and have potential for centrally mediated, undesirable side-effects. Naloxone and naltrexone are examples. The present invention involves the art recognized group of compounds known as peripheral opioid antagonists.

In preferred form, the methods of the present invention involve administering to a patient a compound which is a peripheral mu opioid antagonist compound. The term peripheral designates that the compound acts primarily on physiological systems and components external to the central nervous system, i.e., the compound does not readily cross the blood-brain barrier. The peripheral mu opioid antagonist compounds employed in the methods of the present invention typically exhibit high levels of activity with respect to gastrointestinal tissue, while exhibiting reduced, and preferably substantially no, central nervous system (CNS) activity. The term “substantially no CNS activity”, as used herein, means that less than about 20% of the pharmacological activity of the peripheral mu opioid antagonist compounds employed in the present methods is exhibited in the CNS. In preferred embodiments, the peripheral mu opioid antagonist compounds employed in the present methods exhibit less than about 5% of their pharmacological activity in the CNS, with about 1% or less (i.e., no CNS activity) being still more preferred.

The peripheral opioid antagonist may be, for example, a piperidine-N-alkylcarboxylate such as described in U.S. Pat. Nos. 5,250,542; 5,434,171; 5,159,081; 5,270,328; and 6,469,030. It also may be an opium alkaloid derivative such as described in U.S. Pat. Nos. 4,730,048; 4,806,556; and 6,469,030. Other peripheral opioid antagonists include quaternary benzomorphan compounds such as described in U.S. Pat. Nos. 3,723,440 and 6,469,030. The preferred antagonists are quaternary derivatives of noroxymorphone such as methylnaltrexone, described in U.S. Pat. Nos. 4,176,186 and 5,972,954. Other examples of quaternary derivatives of noroxymorphone include methylnaloxone, and methylnalorphine. All of the foregoing patents are incorporated herein by reference in their entirety.

A particularly preferred quaternary derivative of noroxymorphone is methylnaltrexone and salts thereof, described first by Goldberg, et al. Methylnaltrexone is also described in U.S. Pat. Nos. 4,719,215; 4,861,781; 5,102,887; 6,274,591; U.S. Patent Application Nos. 2002/0028825 and 2003/0022909; and PCT publication Nos. WO 99/22737 and WO 98/25613; each hereby incorporated by reference. As used herein, “methylnaltrexone” includes N-methylnaltrexone and salts thereof.

Methylnaltrexone is provided as a white crystalline powder freely soluble in water. Its melting point is 254-256° C. Methylnaltrexone is available in a powder form from Mallinckrodt Pharmaceuticals, St. Louis, Mo. The compound as provided is 99.4% pure by reverse phase HPLC, and contains less than 0.011% unquaternized naltrexone by the same method. Methylnaltrexone is also identified as N-methyl-naltrexone bromide, N-methylnaltrexone, MNTX, SC-37359, MRZ-2663-BR, naltrexone methobromide, and N-cyclopropylmethylnoroxy-morphine-methobromide.

In one aspect of the invention, the methods of treating IBS comprise administering a peripheral opioid antagonist and at least one IBS therapeutic agent that is not an opioid agonist or peripheral opioid antagonist to a patient suffering from IBS. IBS therapeutic agents include, but are not limited to, benzodiazepine compounds, antispasmodic, selective serotonin reuptake inhibitors (SSRIs), cholecystokinin (CCK) receptor antagonists, motilin receptor agonists or antagonists, natural killer (NK) receptor antagonists, corticotropin Releasing Factor (CRF) receptor agonists or antagonists, somatostatin receptor agonists, antacids, GI relaxants, anti-gas compounds, bismuth-containing preparations, pentosan polysulfate, anti-emetic dopamine D2 antagonists, prostaglandin E analogs, gonadotrophin-releasing hormone analogues (leuprolide), corticotrophin-1 antagonists, neurokinin 2 receptor antagonists, cholecystokinin-1 antagonists, beta-blockers, anti-esophageal reflux agents, anti-muscarinics, antidiarrheals, antiinflammatory agents, pro-motility agents, 5HT₁ agonists, 5HT₃ antagonists, 5HT₄ antagonists, 5HT₄ agonists, bile salt sequestering agents, bulk-forming agents, bulk-forming laxatives, cathartic laxatives, diphenylmethane laxatives, osmotic laxatives, saline laxatives, other laxatives, stool softeners, alpha₂-adrenergic agonists, mineral oils, antidepressants, herbal medicines, juices, fruits, vegetables, and herbal and vegetable juices. In another embodiment, the peripheral opioid antagonist is administered in a formulation comprising the peripheral opioid antagonist and an antibiotic. As used herein, an IBS therapeutic agent specifically excludes peripheral opioid antagonists and opioid agonists.

In some embodiments of the invention, the opioid antagonist is administered in a formulation comprising the peripheral opioid antagonist and one or more IBS therapeutic agents. These formulations may be parenteral or oral, such as the formulations described in U.S. Pat. Nos. 6,277,384; 6,261,599; 5,958,452; and PCT publication No. WO 98/25613, each hereby incorporated by reference. Included are solid, semisolid, liquid, controlled release and other such formulations.

Examples of IBS therapeutic agents according to the invention include, but are not limited to, the following:

Benzodiazepine compounds and analogs which act to suppress seizures through an interaction with y-aminobutyric acid (GABA) receptors of the A-type (GABA_{A}), for example, DIASTAT® and VALIUM®; LIBRIUM®; and ZANAX®.

SSRIs, for example, fluvoxamine; fluoxetine; paroxetine; sertraline; citalopram; venlafaxine; cericlamine; duloxetine; milnacipran; nefazodone; and cyanodothiepin (See The Year Drugs News, 1995 Edition, pp. 47-48 by Prous J. R.) and WO 97/29739.

CCK receptor antagonists, for example, devazepide; lorglumide; dexioxiglumide; loxiglumide, D'Amato, M. et al., Br. J. Pharmacol. Vol. 102(2), pp. 391-395 (1991); C1 988; L364,718; L3637260; L740,093 and LY288,513; CCK receptor antagonists disclosed in U.S. Pat. No. 5,220,017, Bruley-Des-Varannes, S, et al. Gastroenterol. Clin. Biol. Vol.15.(10)9 pp. 744-757 (1991), and Worker C: EUPHAR'99—Second European Congress of Pharmacology (Part IV) Budapest, Hungary Iddb Meeting Report 1999 July 3-7.

Motilin receptor agonists or antagonists which include e.g. motilin agonist ABT-269, (erythromycin, 8,9-didehydro-N-dimethyl deoxo-4″,6,12-trideoxy-6,9-epoxy-N-ethyl), de(Nmethyl-N-ethyl-8,9-anhydroerythromycin A) and de(N-methyl)-N-isoprop-8,9anhydroerythromycin A), Sunazika T. et al., Chem. Pharm. Bull., Vol.37(10), pp. 2687-2700 (1989); A-173508 (Abbot Laboratories); motilin antagonists (Phe3, Leu-13) porcine motilin, 214^{th} American Chemical Society (ACS) Meeting (Part V); Highlights from Medicinal Chemistry Poster Session, Wednesday 1 0 September, Las Vegas, Nev., (1997), Iddb Meeting Report September 7-11 (1997); and ANQ-1 1 125, Peeters T. L., et al., Biochem. Biophys. Res. Commun., Vol. 198(2), pp. 411-416 (1994).

NK receptor antagonists which include e.g. FK 888( Fujisawa); GR 205171 (Glaxo Wellcome); LY 303870 (Lilly); MK 869 (Merck); GR82334 (Glaxo Wellcome); L758298 (Merck); L 733060 (Merck); L 741671 (Merck); L 742694 (Merck); PD 154075 (Parke-Davis); S1 8523 (Servier); S1 9752 (Servier); OT 7100 (Otsuka); WIN 51708 (Sterling Winthrop); NKP-608A; TKA457; DNK333; CP-96345; CP-99994; CP122721; L-733060; L-741671; L742694; L-758298; L-754030; GR-203040; GR-205171; RP-67580; RPR-100893 (dapitant); RPR-107880; RPR-111905; FK-888; SDZ-NKT-343; MEN-10930; MEN-11149; S-18523; S-19752; PD-154075 (CAM-4261); SR-140333; LY-303870 (lanepitant); EP-00652218; EP00585913; L-737488; CGP-49823; WIN-51708; SR-48968 (saredutant); SR-144190; YM383336; ZD-7944; MEN-10627; GR-159897; RPR-106145; PD-147714 (CAM-2291); ZM253270; FK-224; MDL-1 05212A; MDL-105172A; L-743986; L-743986 analogs; S-16474; SR-1 42801 (osanetant); PD-161182; SB-223412; and SB-222200.

CRF receptor agonists or antagonists, e.g. as disclosed in WO 99/40089, AXC 2219, Antalarmin, NGD 1, CRA 0165, CRA 1000, CRA 1001.

Somatostatin receptor agonists, e.g. octreotide, vapreotide, lanreotide.

Anti-inflammatory compounds, particularly those of the immuno-modulatory type, for example, NSAIDS; Tumor Necrosis Factor (TNF, TNFa) inhibitors; basiliximab (e.g. SIMULECT®); daclizumab (e.g. ZENAPAX®); infliximab (e.g. REMICADE®); mycophenolate mofetil (e.g. CELLCEPT®); azathioprine (e.g. IMURAN®); tacrolimus (e.g. PROGRAF®); steroids; and GI anti-inflammatory agents, for example, sulfasalazine (e.g. AZULFIDINE®); olsalazine (e.g. DIPENTUM®); and mesalamine (e.g. ASACOL®, PENTASA®, ROWASA®).

Antacids, such as aluminum and magnesium antacids; and calcium hydroxides such as MAALOX®.

GI relaxants, for example, cholestyramine resin marketed under the trade name LOCHOLEST® and QUESTRAN®.

Anti-gas compounds, for example, simethicone marketed under the trade names MYLANTA® and MYLICON®; and enzyme preps including PHAZYME® and BEANO®.

Bismuth-containing preparations, for example, bismuth subsalicylate also known as PEPTO-BISMOL®.

Pentosan polysulfate, a heparin-like macromolecular carbohydrate derivative which chemically and structurally resembles glycosaminoglycans, marketed under the trade name ELMIRON®.

Anti-emetic dopamine D2 antagonists which include e.g. domperidone.

Prostaglandin E analogs, gonadotrophin-releasing hormone analogues (leuprolide), corticotrophin-1 antagonists, neurokinin 2 receptor antagonists, cholecystokinin-1 antagonists, beta-blockers.

Anti-esophageal reflux agents include but are not limited to PRILOSEC®.

Antispasmodics and anti-muscarinics include, but are not limited to, dicyclomine, oxybutyin (e.g., oxybutynin chloride), tolterodine (e.g., tolterodine tartarate), alverine anisotropine, atropine (e.g., atropine sulfate), belladonna, homatropine, homatropine methobromide, hyoscyamine (e.g., hyoscyamine sulfate), methscopolamine, scopolamine (e.g., scopolamine hydrochloride), clidinium, cimetropium, hexocyclium, pinaverium, otilonium, glycopyrrolate, and mebeverine.

Antidiarrheals include, but are not limited to, ipratropium, isopropamide, mepenzolate, propantheline, oxyphencylcimine, pirenzepine, diphenoxylate (e.g., diphenoxylate hydrochloride), atropine sulfate, alosetron hydrochloride, difenoxin hydrochloride, bismuth subsalicylate, lactobacillus acidophilus, trimebutine, asimadoline, and octreotide acetate.

Antiinflammatory agents include, but are not limited to, mesalamine, sulfasalazine, balsalazide disodium, hydrocortisone, and olsalazine sodium.

Pro-motility agents include, but are not limited to, metaclopramide and cisapride.

5HT₁ agonists include, but are not limited to, buspirone.

5HT₃ antagonists include, but are not limited to, ondansetron, cilansetron, and alosetron.

5HT₄ antagonists include, but are not limited to, piposcrod.

5HT₄ agonists include, but are not limited to, tegaserod (e.g., tegaserod maleate), and povcalopride.

Bile salt sequestering agents include, but are not limited to, cholestyramine.

Bulk-forming agents and bulk-forming laxatives include, but are not limited to, psyllium, methylcellulose, psyllium husks and related preparations and extracts of species of the genus Plantago, plantago hydrocolloid, including psyllium hydrophilic mucilloid, oat hull fiber, oats, senna, cassia pod fiber, sennosides, carboxymethylcellulose, karaya and related preparations from species of the genuses Sterculia or Cochlospermum and malt soup extract.

Cathartic laxatives include, but are not limited to, aloe and related preparations and extracts from species of the genus Aloe, cascara sagrada and related preparations and extracts of the species Rhamnus purshiana such as casanthranol, frangula and related preparations and extracts of the species Rhamnus frangula, senna and related preparations and extracts of species of the genus Cassia, sennosides A and B and combinations thereof and combinations of the above.

Diphenylmethane laxatives include, but are not limited to, bisacodyl, bisacodyl tannex, phenolphthalein, dephenylmethane derivatives, combinations of the above with magnesium salts such as magnesium citrate and combinations of the above with sodium phosphate buffers.

Osmotic laxatives include, but are not limited to, lactulose, sorbitol (d-glucitol), polyethylene glycol solution, and glycerin (glycerol).

Saline laxatives include, but are not limited to, magnesium citrate, magnesium hydroxide, magnesium sulfate, magnesium oxide, sodium phosphate, mono- and di-basic sodium phosphate, potassium bitartrate, sodium bicarbonate, and carbon dioxide releasing agents.

Other laxatives include, but are not limited to, sennoids, casanthanol, docusate sodium, bisacodyl, lactulose, synthetic disaccharides, colonic acidifier which promotes laxation, polyethylene glycols, polyethylene glycol 3350, guiafensin, poloxamer 188 (a copolymer consisting of poly(ethylene oxide)-poly(propylene oxide)-poly(ethylene oxide) in a weight ratio of approximately 4:2:4), 1,8-dihydroxyanthraquinone, herbal teas, polycarbophil, soy milk, caffeine, bentonite clay, castor oil, dehydrocholic acid, and dietary fiber.

Stool softeners include, but are not limited to, docusate, such as docusate calcium (dioctyl calcium sulfosuccinate), docusate potassium (dioctyl potassium sulfosuccinate), and docusate sodium.

Alpha₂-adrenergic agonists include, but are not limited to, clonidine.

Mineral oils include, but are not limited to, heavy liquid petrolatum, heavy mineral oil, liquid paraffin, and white mineral oil. Other oils include, but are not limited to, virgin coconut oil.

Antidepressants include, but are not limited to, desiprimine, amitryptiline, imiprimine, fluoxetine, and paroxetine.

Herbal medicines, juices, fruits, vegetables, and herbal and vegetable juices, juices, fruits, vegetables, and herbal and vegetable juices include, but are not limited to: aloe (aloe, various), hops (Bryonia alba), buckthorn (Rhamnus catharticus), cascara sagrada (Rhamnus purshianus), crampbark (Viburnum opulus), dandelion root (Taraxacum officinale), fenugreek (Trigonella foenum-graecum), flax (Linum usitatissumum), frangula (Frangula alnus), ginger (Zingiber officinale), goldenseal (Hydrastis canadensis), kelp (Fucus sp.), licorice (Glycyrrhiza glabra), nux (Strychnos nux-vomica), lycopodium (Lycopodium sp.), platina psyllium or ispaghula (Plantago sp.), rhubarb (Rheum sp.), senna (Cassia senna), slippery elm (Ulmus rubra), St. John's wort (Hypericum perforatum), yellow dock (Rumex crispus), apple juice, asparagus juice, jicama juice, pear juice, potato juice, prune juice, almond, apple, fig, mango, papaya, parsley, persimmon, pineapple, prune, rutabaga, soybean, tamarind, turnip, walnut, watercress, aconite (Aconitum napellus), agrimony (Agrimonia eupatoria), bael (Aegle marmelos), bistort (Polygonum bistorta), belladona (Atropa belladonna), black catechu (Acacia catechu), bryonia (Bryonia alba), carob (Ceratonia siliqua), chamomile (Chamomilla recutita or Chamaemelum nobile), colocynth (Colocynth cucumis), comfrey (Symphytum officinale), echinacea (Echinacea sp.), fenugreek (Trigonella foenum-graecum), hyoscyamus (Hyoscyamus sp.), ipecac (Cephaelis ipecacuanha), oak (Quercus, various), peppermint or mint (Mentha sp.), psyllium (Plantago sp.), marshmallow root (Athaea officinalis), pulsatilla (anemone plant), sage (Salvia officinalis), sumac (Rhus sp.), tea (Camellia sinensis), valerian (Valerianna oficinalis), veratrum (Veratrum viride), wild yam (Dioscorea villosa), apple (Malus domestica), bayberry (Myrica cerifera), bilberry or blueberry (Vaccinium sp.), blackberry and raspberry (Rubus sp.), carrot (Daucus carota), pomegranate (Punica granatum), yin chen (capillary artemisia leaf), bai zhu (atracylodes root), wu wei zi (schisandra fruit), yi yi ren (Job's tears seed), dang shen (codonopsis root), huo xiang (agastache leaf), chai hu (Chinese thoroughwax root), qin pi (fraxinus chinensis bark), fu ling (wolfporia cocos), che qian zi (asian psyllium seed), huang bai (phellodendron bark), zhi gan cao (licorice root), pao jiang (ginger root), huo po (magnolia bark), fang feng (fang feng root), chen pi (tangerine peel), bai shao (white peony root), mu xiang (costus root), huang lian (Chinese goldthread root), and bai zhi (fragrant angelica root).

Other IBS therapeutic agents include dexioxiglumide, TAK-637, talnetant, SB 223412, AU 244, neurotrophin-3, GT 160-246, immunoglobulin (IgG), ramoplanin, risaxmin, rimethicone, darifenacine, zamifenacin, loxiglumide, misoprostil, leuprolide, domperidone, somatostatin analogues, phenytoin, NBI-34041, saredutant, and dexloxiglumide.

Antibiotics include, but are not limited to, tetracycline antibiotics, such as chlortetracycline, oxytetracycline, tetracycline, demethylchlortetracycline, metacycline, doxycycline, minocycline and rolitetracycline; such as kanamycin, amikacin, gentamicin C₁ₐ, C₂, C_{2b} or C₁, sisomicin, netilmicin, spectinomycin, streptomycin, tobramycin, neomycin B, dibekacin and kanendomycin; macrolides, such as maridomycin and erythromycin; lincomycins, such as clindamycine and lincomycin; penicillanic acid (6-APA)- and cephalosporanic acid (7-ACA)-derivatives having (6β- or 7β-acylamino groups, respectively, which are present in fermentatively, semi-synthetically or totally synthetically obtainable 6β-acylaminopenicillanic acid or 7β-acylaminocephalosporanic acid derivatives and/or 7β-acylaminocephalosporanic acid derivatives that are modified in the 3-position, such as penicillanic acid derivatives that have become known under the names penicillin G or V, such as phenethicillin, propicillin, nafcillin, oxycillin, cloxacillin, dicloxacillin, flucloxacillin, cyclacillin, epicillin, mecillinam, methicillin, azlocillin, sulbenicillin, ticarcillin, mezlocillin, piperacillin, carindacillin, azidocillin or ciclacillin, and cephalosporin derivatives that have become known under the names cefaclor, cefuroxime, cefazlur, cephacetrile, cefazolin, cephalexin, cefadroxil, cephaloglycin, cefoxitin, cephaloridine, cefsulodin, cefotiam, ceftazidine, cefonicid, cefotaxime, cefmenoxime, ceftizoxime, cephalothin, cephradine, cefamandol, cephanone, cephapirin, cefroxadin, cefatrizine, cefazedone, ceftrixon and ceforanid; and other β-lactam antibiotics of the clavam, penem and carbapenen type, such as moxalactam, clavulanic acid, nocardicine A, sulbactam, aztreonam and thienamycin; and other antibiotics including bicozamycin, novobiocin, chloramphenicol or thiamphenicol, rifampicin, fosfomycin, colistin, and vancomycin.

The peripheral opioid antagonist also may be administered together with loperamide, which is an opioid agonist that is an anti-diarrheal. It may be administered with other opioid agonists including, but are not limited to, alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, opium, oxycodone, oxymorphone, pentazocine, propiram, propoxyphene, remifentanyl, sufentanil, tilidine, trimebutine, and tramadol.

An amount effective to treat IBS, as used herein, means that amount necessary to delay the onset of, inhibit the progression of, halt altogether the onset of, halt altogether the progression of, or ameliorate at least one or more symptoms of IBS. By ameliorate at least one symptom of, is meant a patient perceived and/or clinically measurable improvement of one or more symptoms of IBS, a lessening of the severity of one or more symptoms, or to make more tolerable one or more symptoms of IBS.

Generally, oral doses of the quaternary derivatives of noroxymorphone will be from about 0.25 to about 5.0 mg/kg body weight per day. It is expected that oral doses in the range from 0.5 to 5.0 mg/kg body weight will yield the desired results. Generally, parenteral administration, including intravenous and subcutaneous administration, will be from about 0.001 to 1.0 mg/kg body weight. It is expected that doses ranging from 0.001 to 0.45 mg/kg body weight will yield the desired results, and doses of 0.1 to 0.3 are preferred. It is expected that infusion doses in the range from 0.001 to 1 mg/kg body weight will yield the desired results. Dosage may be adjusted appropriately to achieve desired drug levels, local or systemic, depending on the mode of administration. For example, it is expected that the dosage for oral administration of the opioid antagonists in an enterically-coated formulation would be from 10 to 30% of the non-coated oral dose. In the event that the response in a patient is insufficient at such doses, even higher doses (or effectively higher dosage by a different, more localized delivery route) may be employed to the extent that the patient tolerance permits. Oral administration may also include colonic site-directed release formulations. Multiple doses per day are contemplated to achieve appropriate systemic levels of compounds. Appropriate system levels can be determined by, for example, measurement of the patient's peak or sustained plasma level of the drug. “Dose” and “dosage” are used interchangeably herein.

The formulations can be constructed and arranged to create mean peak plasma levels. Mean peak plasma concentrations can be measured using HPLC techniques, as are known to those of skill in the art. Mean peak (i.e., steady state) is achieved when the rate of drug availability is equal to the rate of drug elimination from the circulation. In typical therapeutic settings, the quaternary derivatives of noroxymorphone will be administered to patients either on a periodic dosing regimen or with a constant infusion regimen. The concentration of drug in the plasma will tend to rise immediately after the onset of administration and will tend to fall over time as the drug is eliminated from the circulation by means of distribution into cells and tissues, by metabolism, or by excretion. Mean peak will obtain when the mean drug concentration remains constant over time. In the case of intermittent dosing, the pattern of the drug concentration cycle is repeated identically in each interval between doses with the mean concentration remaining constant. In the case of constant infusion, the mean drug concentration will remain constant with very little oscillation. The achievement of steady state is determined by means of measuring the concentration of drug in plasma over at least one cycle of dosing such that one can verify that the cycle is being repeated identically from dose to dose. Typically, in an intermittent dosing regimen, maintenance of steady state can be verified by determining drug concentrations at the consecutive troughs of a cycle, just prior to administration of another dose. In a constant infusion regimen where oscillation in the concentration is low, steady state can be verified by any two consecutive measurements of drug concentration. “Mean peak” and “steady state” are used interchangeably herein.

A variety of administration routes are available. The particular mode selected will depend, of course, upon the particular combination of drugs selected, the severity of the IBS being treated, or prevented, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, sublingual, transdermal, intravenous infusion, pulmonary, intramuscular, intracavity, aerosol, aural (e.g., via eardrops), intranasal, inhalation, needleless injection, or subcutaneous delivery. Direct injection could also be preferred for local delivery. For continuous infusion, a PCA device may be employed. Oral or subcutaneous administration may be important for prophylactic or long-term treatment because of the convenience of the patient as well as the dosing schedule. Preferred rectal modes of delivery include administration as a suppository or enema wash. For transdermal administration, an ionopheresis device may be employed to enhance penetration of the active drug through the skin. Such devices and methods useful in ionophoresis current assisted transdermal administration include those described in U.S. Pat. Nos. 4,141,359; 5,499,967; and 6,391,015.

The pharmaceutical preparations may conveniently be presented in unit dosage form and may be prepared by any of the methods well known in the art of pharmacy. All methods include the step of bringing the compounds of the invention into association with a carrier which constitutes one or more accessory ingredients. In general, the compositions are prepared by uniformly and intimately bringing the compounds of the invention into association with a liquid carrier, a finely divided solid carrier, or both, and then, if necessary, shaping the product.

When administered, the pharmaceutical preparations of the invention are applied in pharmaceutically acceptable compositions. Such preparations may routinely contain salts, buffering agents, preservatives, compatible carriers, lubricants and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluenesulfonic, tartaric, citric, methanesulfonic, formic, succinic, naphthalene-2-sulfonic, pamoic, 3-hydroxy-2-naphthalenecarboxylic, and benzene sulfonic.

The pharmaceutical preparations of the present invention may include or be diluted into a pharmaceutically-acceptable carrier. The term “pharmaceutically-acceptable carrier” as used herein means one or more compatible solid or liquid filler, diluents or encapsulating substances which are suitable for administration to a human or other mammal such as a dog, cat, horse, cow, sheep, or goat. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carrier formulations suitable for oral administration, for suppositories, and for parenteral administration, etc., can be found in Remington's Pharmaceutical Sciences, Mack Publishing Company, Easton, Pa.

The pharmaceutical preparations of the invention, as well as the pharmaceutical preparations that are administered to treat IBS, are free of bioavailable calcium and bioavailable calcium salts. “Free of calcium,” as used herein, means that calcium, including ions thereof, is present in the pharmaceutical preparation in a concentration of 1% or less. In some embodiments, there may be less than 0.5%, 0.1%, 0.01%, 0.001%, and even 0.0001%. Preferably, there is no detectable level of calcium present. In particular, the pharmaceutical preparations of the present invention are free of exogenously or intentionally added bioavailable calcium and bioavailable calcium salts such as soluble calcium salts including ascorbate, gluconate, glucoheptonate, dobesilate, glucobionate, levulinate, lactate, lactobionate, pantotenate, ketoglutarate, borogluconate, and the like.

Aqueous formulations may include one or more of a chelating agent, a buffering agent, an anti-oxidant, an isotonicity agent, and a preservative. In the case of quaternary amine derivatives of noroxymorphone, a chelating agent can be added and pH can be adjusted to between 3.0 and 3.5. Preferred such formulations that are stable to autoclaving and long term storage are described in co-pending application Ser. No. 60/461,611, filed on the same date hereof, entitled “Pharmaceutical Formulation”, the disclosure of which is incorporated herein by reference.

Chelating agents include: ethylenediaminetetraacetic acid (EDTA) and derivatives thereof, citric acid and derivatives thereof, niacinamide and derivatives thereof, sodium desoxycholate and derivatives thereof.

Buffering agents include: citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate and benzoic acid, and combinations thereof.

Antioxidants include: those selected from the group consisting of an ascorbic acid derivative, butylated hydroxy anisole, butylated hydroxy toluene, alkyl gallate, sodium meta-bisulfite, sodium bisulfite, sodium dithionite, sodium thioglycollate, sodium formaldehyde sulfoxylate, tocopheral and derivatives thereof, monothioglycerol, and sodium sulfite. The preferred antioxidant is monothioglycerol.

Isotonicity agents include: those selected from the group consisting of sodium chloride, mannitol, lactose, dextrose, glycerol, and sorbitol.

Preservatives that can be used with the present compositions include benzyl alcohol, parabens, thimerosal, chlorobutanol and benzalkonium chloride and preferably benzalkonium chloride is used. Typically, the preservative will be present in a composition in a concentration of up to about 2% by weight. The exact concentration of the preservative, however, will vary depending upon the intended use and can be easily ascertained by one skilled in the art.

The subjects can be treated with a combination of the peripheral opioid antagonist and an IBS therapeutic agent(s) and/or an opioid. In these circumstances the opioid antagonist and the other therapeutic agent(s) are administered close enough in time to have the simultaneous benefit of both agents. In some embodiments the opioid antagonist will be delivered first in time, in some embodiments second in time and still in some embodiments at the same time. The peripheral opioid antagonist and the IBS therapeutic agent(s) and/or an opioid may be administered by the same or different routes of administration. As discussed in greater detail below, the invention contemplates pharmaceutical preparations where the agents are contained in the same pharmaceutical preparation.

A product containing a peripheral opioid antagonist and an IBS therapeutic agent (and/or an opioid) can be configured as an oral dosage. The oral dosage may be a liquid, a semi-solid or a solid. The oral dosage can include the opioid antagonist together with a laxative or a stool softener. An opioid may optionally be included in the oral dosage. The oral dosage may be configured to release the peripheral opioid antagonist before, after or simultaneously with the laxative or stool softener (and/or the opioid). The oral dosage may be configured to have the peripheral opioid antagonist and the other agents release completely in the stomach, release partially in the stomach and partially in the intestine or only in the intestine. The oral dosage also may be configured whereby the release of the peripheral opioid antagonist is confined to the stomach or intestine while the release of the other active agent is not so confined or is confined differently from the peripheral opioid antagonist. For example, the peripheral opioid antagonist may be an enterically coated core or pellets contained within a pill or capsule that releases the other agent(s) first and releases the peripheral opioid antagonist only after the peripheral opioid antagonist passes through the stomach and into the intestine. The peripheral opioid antagonist also can be in a sustained release material, whereby the peripheral opioid antagonist is released throughout the gastrointestinal tract and the other agent(s) is released on the same or a different schedule. The same objective for peripheral opioid antagonist release can be achieved with immediate release of peripheral opioid antagonist combined with enteric coated opioid antagonist. In these instances, the other agent(s) could be released immediately in the stomach, throughout the gastrointestinal tract or only in the intestine.

The materials useful for achieving these different release profiles are well known to those of ordinary skill in the art. Immediate release is obtainable by conventional tablets with binders which dissolve in the stomach. Coatings which dissolve at the pH of the stomach or which dissolve at elevated temperatures will achieve the same purpose. Release only in the intestine is achieved using conventional enteric coatings such as pH sensitive coatings which dissolve in the pH environment of the intestine (but not the stomach) or coatings which dissolve over time. Release throughout the gastrointestinal tract is achieved by using sustained-release materials and/or combinations of the immediate release systems and sustained and/or delayed intentional release systems (e.g., pellets which dissolve at different pHs).

A product containing both a peripheral opioid antagonist and an IBS therapeutic agent also can be configured as a suppository. The peripheral opioid antagonist can be placed anywhere within or on the suppository to favorably affect the relative release of the opioid antagonist. The nature of the release can be zero order, first order, or sigmoidal, as desired.

In the event that it is desirable to release the peripheral opioid antagonist first, the peripheral opioid antagonist could be coated on the surface of the suppository in any pharmaceutically acceptable carrier suitable for such coatings and for permitting the release of the peripheral opioid antagonist, such as in a temperature sensitive pharmaceutically acceptable carrier used for suppositories routinely. Other coating which dissolve when placed in a body cavity are well known to those of ordinary skill in the art.

The peripheral opioid antagonist also may be mixed throughout the suppository, whereby it is released before, after or simultaneously with the other agent(s). The peripheral opioid antagonist may be free, that is, solubilized within the material of the suppository. The peripheral opioid antagonist also may be in the form of vesicles, such as wax coated micropellets dispersed throughout the material of the suppository. The coated pellets can be fashioned to immediately release the peripheral opioid antagonist based on temperature, pH or the like. The pellets also can be configured so as to delay the release of the peripheral opioid antagonist, allowing the other agent(s) a period of time to act before the peripheral opioid antagonist exerts its effects. The peripheral opioid antagonist pellets also can be configured to release the peripheral opioid antagonist in virtually any sustained release pattern, including patterns exhibiting first order release kinetics or sigmoidal order release kinetics using materials of the prior art and well known to those of ordinary skill in the art.

The peripheral opioid antagonist also can be contained within a core within the suppository. The core may have any one or any combination of the properties described above in connection with the pellets. The peripheral opioid antagonist may be, for example, in a core coated with a material, dispersed throughout a material, coated onto a material or adsorbed into or throughout a material.

It should be understood that the pellets or core may be of virtually any type. They may be drug coated with a release material, drug interspersed throughout material, drug adsorbed into a material, and so on. The material may be erodible or nonerodible.

The oral product or suppository optionally can contain an opioid. The opioid can be in any of the forms described above in connection with the peripheral opioid antagonist, but separate from the peripheral opioid antagonist. The opioid also may be mixed together with the peripheral opioid antagonist and provided in any of the forms described above in connection with peripheral opioid antagonist.

Any of the active agents (i.e., ingredients) may be provided in particles. Particles as used herein means nano or microparticles (or in some instances larger) which consist in whole or in part of the peripheral opioid antagonists or other therapeutic agent(s) as described herein. The particles may contain the active ingredients in a core surrounded by a coating, including, but not limited to, an enteric coating. The active ingredients also may be dispersed throughout the particles. The active ingredients also may be adsorbed into the particles. The particles may be of any order release kinetics, including zero order release, first order release, second order release, delayed release, sustained release, immediate release, and any combination thereof, etc. The particle may include, in addition to the active ingredients, any of those materials routinely used in the art of pharmacy and medicine, including, but not limited to, erodible, nonerodible, biodegradable, or nonbiodegradable material or combinations thereof. The particles may be microcapsules which contain the antagonist in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as nonimmediate release formulations, with nonimmediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.” Delivery systems specific for the gastrointestinal tract are roughly divided into three types: the first is a delayed release system designed to release a drug in response to, for example, change in pH or temperature; the second is a timed-release system designed to release a drug after a predetermined time; and the third is a microflora enzyme system making use of the abundant enterobacteria in the lower part of the gastrointestinal tract.

An example of a delayed release system is one that uses, for example, an acrylic or cellulosic coating material and dissolves on pH change. Because of ease of preparation, many reports on such “enteric coatings” have been made. In general, an enteric coating is one which passes through the stomach without releasing substantial amounts of drug in the stomach (i.e., less than 10% release, 5% release and even 1% release in the stomach) and sufficiently disintegrating in the intestine tract (by contact with approximately neutral or alkaline intestine juices) to allow the transport (active or passive) of the active agent through the walls of the intestinal tract.

Various in vitro tests for determining whether or not a coating is classified as an enteric coating have been published in the pharmacopoeia of various countries. A coating which remains intact for at least 2 hours, in contact with artificial gastric juices such as HCl of pH 1 at 36 to 38° C. and thereafter disintegrates within 30 minutes in artificial intestinal juices such as a KH₂PO₄ buffered solution of pH 6.8 is one example. One such well known system is EUDRAGIT material, commercially available and reported on by Behringer, Manchester University, Saale Co., and the like. Enteric coatings are discussed further, below.

A timed release system is represented by Time Erosion System (TES) by Fujisawa Pharmaceutical Co., Ltd. and Pulsincap by R. P. Scherer. According to these systems, the site of drug release is decided by the time of transit of a preparation in the gastrointestinal tract. Since the transit of a preparation in the gastrointestinal tract is largely influenced by the gastric emptying time, some time release systems are also enterically coated.

Systems making use of the enterobacteria can be classified into those utilizing degradation of azoaromatic polymers by an azo reductase produced from enterobacteria as reported by the group of Ohio University (M. Saffran et al., Science, Vol. 233: 1081 (1986)) and the group of Utah University (J. Kopecek et al., Pharmaceutical Research, 9(12), 1540-1545 (1992)); and those utilizing degradation of polysaccharides by beta-galactosidase of enterobacteria as reported by the group of Hebrew University (unexamined published Japanese patent application No. 5-50863 based on a PCT application) and the group of Freiberg University (K. H. Bauer et al., Pharmaceutical Research, 10(10), S218 (1993)). In addition, the system using chitosan degradable by chitosanase by Teikoku Seiyaku K. K. (unexamined published Japanese patent application No. 4-217924 and unexamined published Japanese patent application No. 4-225922) is also included.

The enteric coating is typically although not necessarily a polymeric material. Preferred enteric coating materials comprise bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers such as cellulose acetate phthalate, cellulose acetate trimellitate, hydroxypropylmethyl cellulose phthalate, hydroxypropyhnethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl methacrylate (e.g., those copolymers sold under the tradename “EUDRAGIT”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). Combinations of different coating materials may also be used. Well known enteric coating material for use herein are those acrylic acid polymers and copolymers available under the tradename EUDRAGIT from Rohm Pharma (Germany). The EUDRAGIT series E, L, S, RL, RS and NE copolymers are available as solubilized in organic solvent, as an aqueous dispersion, or as a dry powder. The EUDRAGIT series RL, NE, and RS copolymers are insoluble in the gastrointestinal tract but are permeable and are used primarily for extended release. The EUDRAGIT series E copolymers dissolve in the stomach. The EUDRAGIT series L, L-30D and S copolymers are insoluble in stomach and dissolve in the intestine, and are thus most preferred herein.

A particular methacrylic copolymer is EUDRAGIT L, particularly L-30D and EUDRAGIT L100-55. In EUDRAGIT L-30D, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another particular methacrylic acid polymer is EUDRAGIT S, which differs from EUDRAGIT L-30D in that the ratio of free carboxyl groups to ester groups is approximately 1:2. EUDRAGIT S is insoluble at pH below 5.5, but unlike EUDRAGIT L-30D, is poorly soluble in gastrointestinal fluids having a pH in the range of 5.5 to 7.0, such as in the small intestine. This copolymer is soluble at pH 7.0 and above, i.e., the pH generally found in the colon. EUDRAGIT S can be used alone as a coating to provide drug delivery in the large intestine. Alternatively, EUDRAGIT S, being poorly soluble in intestinal fluids below pH 7, can be used in combination with EUDRAGIT L-30D, soluble in intestinal fluids above pH 5.5, in order to provide a delayed release composition which can be formulated to deliver the active agent to various segments of the intestinal tract. The more EUDRAGIT L-30D used, the more proximal release and delivery begins, and the more EUDRAGIT S used, the more distal release and delivery begins. It will be appreciated by those skilled in the art that both EUDRAGIT L-30D and EUDRAGIT S can be replaced with other pharmaceutically acceptable polymers having similar pH solubility characteristics.

In certain embodiments of the invention, the preferred enteric coating is ACRYL-EZE™ (methacrylic acid copolymer type C; Colorcon, West Point, Pa.).

The enteric coating provides for controlled release of the active agent, such that drug release can be accomplished at some generally predictable location. The enteric coating also prevents exposure of the therapeutic agent and carrier to the epithelial and mucosal tissue of the buccal cavity, pharynx, esophagus, and stomach, and to the enzymes associated with these tissues. The enteric coating therefore helps to protect the active agent, carrier and a patient's internal tissue from any adverse event prior to drug release at the desired site of delivery. Furthermore, the coated material of the present invention allow optimization of drug absorption, active agent protection, and safety. Multiple enteric coatings targeted to release the active agent at various regions in the gastrointestinal tract would enable even more effective and sustained improved delivery throughout the gastrointestinal tract.

The coating can, and usually does, contain a plasticizer to prevent the formation of pores and cracks that would permit the penetration of the gastric fluids. Suitable plasticizers include, but are not limited to, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will usually contain approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating can be applied to particles of the therapeutic agent(s), tablets of the therapeutic agent(s), capsules containing the therapeutic agent(s) and the like, using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman, et al. (New York: Marcel Dekker, Inc., 1989), and in Ansel, et al., Pharmaceutical Dosage Forms and Drug Delivery Systems, 6^{th} Ed. (Media, Pa.: Williams & Wilkins, 1995). The coating thickness, as noted above, must be sufficient to ensure that the oral dosage form remains intact until the desired site of topical delivery in the lower intestinal tract is reached.

In another embodiment, drug dosage forms are provided that comprise an enterically coated, osmotically activated device housing a formulation of the invention. In this embodiment, the drug-containing formulation is encapsulated in a semipermeable membrane or barrier containing a small orifice. As known in the art with respect to so-called “osmotic pump” drug delivery devices, the semipermeable membrane allows passage of water in either direction, but not drug. Therefore, when the device is exposed to aqueous fluids, water will flow into the device due to the osmotic pressure differential between the interior and exterior of the device. As water flows into the device, the drug-containing formulation in the interior will be “pumped” out through the orifice. The rate of drug release will be equivalent to the inflow rate of water times the drug concentration. Suitable materials for the semipermeable membrane include, but are not limited to, polyvinyl alcohol, polyvinyl chloride, semipermeable polyethylene glycols, semipermeable polyurethanes, semipermeable polyamides, semipermeable sulfonated polystyrenes and polystyrene derivatives; semipermeable poly(sodium styrenesulfonate), semipermeable poly(vinylbenzyltrimethylammonium chloride), and cellulosic polymers such as cellulose acetate, cellulose diacetate, cellulose triacetate, cellulose propionate, cellulose acetate propionate, cellulose acetate butyrate, cellulose trivalerate, cellulose trilmate, cellulose tripalmitate, cellulose trioctanoate, cellulose tripropionate, cellulose disuccinate, cellulose dipalmitate, cellulose acetate succinate, cellulose propionate succinate, cellulose acetate octanoate, cellulose valerate palmitate, cellulose acetate heptanate, cellulose acetaldehyde dimethyl acetate, cellulose acetate ethylcarbamate, cellulose acetate methylcarbamate, cellulose dimethylaminoacetate and ethylcellulose.

Enterically coated, osmotically activated devices can be manufactured using conventional materials, methods and equipment. For example, osmotically activated devices may be made by first encapsulating, in a pharmaceutically acceptable soft capsule, a liquid or semi-solid formulation as described previously. This interior capsule is then coated with a semipermeable membrane composition (comprising, for example, cellulose acetate and polyethylene glycol 4000 in a suitable solvent such as a methylene chloride-methanol admixture), for example using an air suspension machine, until a sufficiently thick laminate is formed, e.g., around 0.05 mm. The semipermeable laminated capsule is then dried using conventional techniques. Then, an orifice having a desired diameter (e.g., about 0.99 mm) is provided through the semipermeable laminated capsule wall, using, for example, mechanical drilling, laser drilling, mechanical rupturing, or erosion of an erodible element such as a gelatin plug. The osmotically activated device may then be enterically coated as previously described. For osmotically activated devices containing a solid carrier rather than a liquid or semi-solid carrier, the interior capsule is optional; that is, the semipermeable membrane may be formed directly around the carrier-drug composition. However, preferred carriers for use in the drug-containing formulation of the osmotically activated device are solutions, suspensions, liquids, immiscible liquids, emulsions, sols, colloids, and oils. Particularly preferred carriers include, but are not limited to, enterically coated capsules containing liquid or semisolid drug formulations.

In another embodiment, drug dosage forms are provided that comprise a sustained release coated device housing a formulation of the invention. In this embodiment, the drug-containing formulation is encapsulated in a sustained release membrane. The membrane may be semipermeable, as described above. Semipermeable membranes allow passage of water inside the coated device and then dissolve the drug. The dissolved drug solution then diffuses out through the semipermeable membrane. The rate of drug release therefore depends upon the thickness of the coated film and the release of drug can begin in any part of the GI tract. Suitable membrane materials include ethyl cellulose.

In another embodiment, drug dosage forms are provided that comprise a sustained release device housing a formulation of the invention. In this embodiment, the drug-containing formulation is uniformly mixed with a sustained release polymer. These sustained release polymers may be high molecular weight water-soluble polymers, which when contacted may be water, swell and create channels for water to diffuse inside and dissolve the drug. As the polymers swell and dissolve in water, more of drug is exposed to water for dissolution. Such a system is generally referred to as a sustained release matrix. Suitable materials for such a system include hydropropyl methylcellulose, hydroxypropyl cellulose, hydroxyethyl cellulose, and methyl cellulose.

In another embodiment, drug dosage forms are provided that comprise an enteric coated device housing a sustained release formulation of the invention. In this embodiment, the drug containing product described above coated with an enteric polymers. Such a device does not release any drug in the stomach. When the device reaches the intestine, the enteric polymer begins to dissolve and release the drug. The drug release may take place in a sustained release fashion.

Cellulose coatings include those of cellulose acetate phthalate and trimellitate; methacrylic acid copolymers, e.g. copolymers derived from methylacrylic acid and esters thereof, containing at least 40% methylacrylic acid; and especially hydroxypropyl methylcellulose phthalate. Methylacrylates include those of molecular weight above 100,000 daltons based on, e.g. methylacrylate and methyl or ethyl methylacrylate in a ratio of about 1:1. Typical products include EUDRAGIT L, e.g. L 100-55, marketed by Rohm GmbH, Darmstadt, Germany. Typical cellulose acetate phthalates have an acetyl content of 17-26% and a phthalate content of from 30-40% with a viscosity of ca. 45-90 cP. Typical cellulose acetate trimellitates have an acetyl content of 17-26%, a trimellityl content from 25-35% with a viscosity of ca. 15-20 cS. An example of a cellulose acetate trimellitate is the marketed product CAT (Eastman Kodak Company, USA). Hydroxypropyl methylcellulose phthalates typically have a molecular weight of from 20,000 to 130,000 daltons, a hydroxypropyl content of from 5 to 10%, a methoxy content of from 18 to 24% and a phthalyl content from 21 to 35%. An example of a cellulose acetate phthalate is the marketed product CAP (Eastman Kodak, Rochester N.Y., USA). Examples of hydroxypropyl methylcellulose phthalates are the marketed products having a hydroxypropyl content of from 6-10%, a methoxy content of from 20-24%, a phthalyl content of from 21-27%, a molecular weight of about 84,000 daltons, known under the trade mark HP50 and available from Shin-Etsu Chemical Co. Ltd., Tokyo, Japan, and having a hydroxypropyl content, a methoxyl content, and a phthalyl content of 5-9%, 18-22% and 27-35%, respectively, and a molecular weight of 78,000 daltons, known under the trademark HP55 and available from the same supplier.

The therapeutic agents may be provided in capsules, coated or not. The capsule material may be either hard or soft, and as will be appreciated by those skilled in the art, typically comprises a tasteless, easily administered and water soluble compound such as gelatin, starch or a cellulosic material. The capsules are preferably sealed, such as with gelatin bands or the like. See, for example, Remington: The Science and Practice of Pharmacy, Nineteenth Edition (Easton, Pa.: Mack Publishing Co., 1995), which describes materials and methods for preparing encapsulated pharmaceuticals.

The therapeutic agents may be provided in suppositories. Suppositories are solid dosage forms of medicine intended for administration via the rectum. Suppositories are compounded so as to melt, soften, or dissolve in the body cavity (around 98.6° F.) thereby releasing the medication contained therein. Suppository bases should be stable, nonirritating, chemically inert, and physiologically inert. Many commercially available suppositories contain oily or fatty base materials, such as cocoa butter, coconut oil, palm kernel oil, and palm oil, which often melt or deform at room temperature necessitating cool storage or other storage limitations. U.S. Pat. No. 4,837,214 to Tanaka, et al. describes a suppository base comprised of 80 to 99 percent by weight of a lauric-type fat having a hydroxyl value of 20 or smaller and containing glycerides of fatty acids having 8 to 18 carbon atoms combined with 1 to 20 percent by weight diglycerides of fatty acids (which erucic acid is an example of). The shelf life of these type of suppositories is limited due to degradation. Other suppository bases contain alcohols, surfactants, and the like which raise the melting temperature but also can lead to poor absorption of the medicine and side effects due to irritation of the local mucous membranes (see for example, U.S. Pat. No. 6,099,853 to Hartelendy et al., U.S. Pat. No. 4,999,342 to Ahmad, et al., and U.S. Pat. No. 4,765,978 to Abidi, et al.).

The base used in the pharmaceutical suppository composition of this invention include, in general, oils and fats comprising triglycerides as main components such as cacao butter, palm fat, palm kernel oil, coconut oil, fractionated coconut oil, lard and WITEPSOL®, waxes such as lanolin and reduced lanolin; hydrocarbons such as VASELINE®, squalene, squalane and liquid paraffin; long to medium chain fatty acids such as caprylic acid, lauric acid, stearic acid and oleic acid; higher alcohols such as lauryl alcohol, cetanol and stearyl alcohol; fatty acid esters such as butyl stearate and dilauryl malonate; medium to long chain carboxylic acid esters of glycerin such as triolein and tristearin; glycerin-substituted carboxylic acid esters such as glycerin acetoacetate; and polyethylene glycols and its derivatives such as macrogols and cetomacrogol. They may be used either singly or in combination of two or more. If desired, the composition of this invention may further include a surface active agent, a coloring agent, etc., which are ordinarily used in suppositories.

The pharmaceutical composition of this invention may be prepared by uniformly mixing predetermined amounts of the active ingredient, the absorption aid and optionally the base, etc. in a stirrer or a grinding mill, if required at an elevated temperature. The resulting composition may be formed into a suppository in unit dosage form by, for example, casting the mixture in a mold, or by forming it into a gelatin capsule using a capsule filling machine.

The compositions according to the present invention also can be administered as a nasal spray, nasal drop, suspension, gel, ointment, cream or powder. The administration of a composition can also include using a nasal tampon or a nasal sponge containing a composition of the present invention.

The nasal delivery systems that can be used with the present invention can take various forms including aqueous preparations, non-aqueous preparations and combinations thereof. Aqueous preparations include, for example, aqueous gels, aqueous suspensions, aqueous liposomal dispersions, aqueous emulsions, aqueous microemulsions and combinations thereof. Non-aqueous preparations include, for example, non-aqueous gels, non-aqueous suspensions, non-aqueous liposomal dispersions, non-aqueous emulsions, non-aqueous microemulsions and combinations thereof. The various forms of the nasal delivery systems can include a buffer to maintain pH, a pharmaceutically acceptable thickening agent and a humectant. The pH of the buffer can be selected to optimize the absorption of the therapeutic agent(s) across the nasal mucosa.

With respect to the non-aqueous nasal formulations, suitable forms of buffering agents can be selected such that when the formulation is delivered into the nasal cavity of a mammal, selected pH ranges are achieved therein upon contact with, e.g., a nasal mucosa. In the present invention, the pH of the compositions should be maintained from about 2.0 to about 6.0. It is desirable that the pH of the compositions is one which does not cause significant irritation to the nasal mucosa of a recipient upon administration.

The viscosity of the compositions of the present invention can be maintained at a desired level using a pharmaceutically acceptable thickening agent. Thickening agents that can be used in accordance with the present invention include methyl cellulose, xanthan gum, carboxymethyl cellulose, hydroxypropyl cellulose, carbomer, polyvinyl alcohol, alginates, acacia, chitosans and combinations thereof. The concentration of the thickening agent will depend upon the agent selected and the viscosity desired. Such agents can also be used in a powder formulation discussed above.

The compositions of the present invention can also include a humectant to reduce or prevent drying of the mucus membrane and to prevent irritation thereof. Suitable humectants that can be used in the present invention include sorbitol, mineral oil, vegetable oil and glycerol; soothing agents; membrane conditioners; sweeteners; and combinations thereof. The concentration of the humectant in the present compositions will vary depending upon the agent selected.

One or more IBS therapeutic agents and/or opioids may be incorporated into the nasal delivery system or any other delivery system described herein.

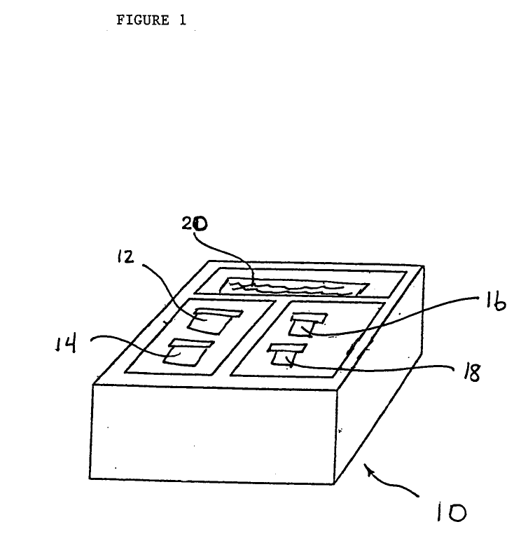

In some aspects of the invention, kits are provided. Referring to FIG. 1, a kit 10 is depicted. The kit 10 includes a pharmaceutical preparation vial 12, a pharmaceutical preparation diluent vial 14, optionally vial 16, and optionally diluent vial 18. The kit also includes instructions 20. The vial 14 containing the diluent for the pharmaceutical preparation is optional. The vial 14 contains a diluent such as physiological saline for diluting what could be a concentrated solution or lyophilized preparation of methylnaltrexone contained in vial 12. The instructions can include instructions for mixing a particular amount of the diluent with a particular amount of the concentrated pharmaceutical preparation, whereby a final formulation for injection or infusion is prepared. The instructions may include instructions for use in a PCA devise. Likewise, the kit optionally contains an antibiotic and/or IBS therapeutic agent antibiotic and/or IBS therapeutic agent in the vial 16, which also optionally may be in a concentrated form. The optional vial 18 contains a diluent for a concentrated antibiotic and/or IBS therapeutic agent. The instructions also may include instructions for mixing the antibiotic and/or IBS therapeutic agent with the pharmaceutical preparation and/or diluting the opioid with the antibiotic and/or IBS therapeutic agent diluent contained in the opioid diluent vial 18. The instructions, therefore, would take a variety of forms depending on the presence or absence of diluent and antibiotic and/or IBS therapeutic agent. The instructions 20 can include instructions for treating a patient with an effective amount of methylnaltrexone. It also will be understood that the containers containing the pharmaceutical preparation, whether the container is a bottle, a vial with a septum, an ampoule with a septum, an infusion bag, and the like, can contain indicia such as conventional markings which change color when the pharmaceutical preparation has been autoclaved or otherwise sterilized.

All of the patents, patent applications and references listed herein are incorporated by reference in their entirety.

### EXAMPLES

The following Examples are intended to illustrate an aspect of the invention and are not to be construed as limitations upon the invention.

### Example 1

### Administration of Methylnaltrexone in Individuals Who Are Not Receiving Opioids

With approval from the Institutional Review Board, 12 normal subjects (8 males and 4 non-pregnant females) participated in a controlled trial. The mean age was 29.3±5.8 (mean+/−standard deviation [SD]) years. None of the subjects had a drug abuse disorder or received any opioids during the trial. Subjects were administered 12 consecutive doses of methylnaltrexone at a dosage rate of 0.3 mg/kg every 6 hours via intravenous injection. Methylnaltrexone was dissolved in isotonic saline for administration in this study. No other excipients were present in the administered solution. Oral-cecal transit time was measured prior to the first dose and after the last dose, following repeated dosing for 3 days, using a lactulose hydrogen breath test (Yuan, C. S., et al., Clin. Pharmacol. Ther. 1996;59:469-475). A subjective rating test for possible opioid agonist effects was also employed (Yuan, C. S., et al., Drug Alcohol Dependence 1998;52:161-165). No significant adverse effects were observed during the study. The results of the oral-cecal transit time tests showed that transit time was reduced from a baseline mean of 101.3±29.4 minutes prior to the first dose of methylnaltrexone to 82.5±20.7 after 3 days of treatment. The reduction in the means was statistically significant at the level of P<0.05 using the paired t-test and the Wilcoxon signed rank test. The overall opioid subjective ratings tended toward a reduction during the 3 days of treatment, but the reductions did not reach statistical significance. These results demonstrate that methylnaltrexone in the absence of calcium ions causes a statistically significant reduction of gut transit time in normal subjects who are not receiving exogenous opioids. The absence of statistically significant changes in overall subjective opioid ratings is consistent with the lack of penetration into the central nervous system by methylnaltrexone. These data suggest that endogenous opioid action is involved regulating human gut motility and that peripheral opioid antagonist can be used to affect favorably gut segmentation and peristalsis, and thereby treating IBS.

### Example 2

### Manufacturing Details for Methylnaltrexone 225 mg Tablets

### (Non-Enteric)

| | mg per tablet |
|---|---|
| Ingredients used (Trade name) | |
| Methylnaltrexone | 225 |
| Microcrystalline cellulose (Avicel PH 101) | 80 |
| Polyvinylpyrrolidone (Povidone K30) | 10.50 |
| Croscarmellose sodium | 8 |
| (Ac-Di-Sol SD-711) | |
| Dibasic Calcium Phosphate (Emcompress) | 25 |
| NO AVICEL PH 200 WAS USED | |
| Magnesium Stearate (Hyqual) | 1.7 |
| Opadry II Clear | 7.00 |
| Water | as needed |
| Equipment used | |
| Key KG-5 Granulator | to make granules . . . kind |
| | of dough maker |
| Glatt WSG-1, Uniglatt | to dry the granules |
| Quadro Comill | to break the granule particles |
| | to the desired size |
| Cross-Flow blender | to mix things together |
| Manesty beta-press | to compress powder |
| | into tablets |
| O'Hara Labcoat II-X | to coat the tablet |
| | s with any film. |

Miscellaneous equipments such as balances, peristaltic pump, propeller mixer and spatula etc.

Manufacturing steps:
1. Pass Methylnaltexone, Avicel 101 and Ac-Di-Sol (part of it) thru 20 mesh screen and add to the granulator.
2. Granulate the above mixture using a solution of Povidone in water.
3. After the granules are formed, transfer the material to Uniglatt and dry the mixture.
4. Repeat steps 1 to 3, EIGHT more times and combine the mixture. This was done due to equipment capacity being 1/9 of the total weight.
5. Pass the mixture in step #4 thru Comill.
6. Screen Avicel 101, Emcompress and the remaining Ac-Di-Sol thru 20 mesh screen and add it to the blender.
7. Add material from step #5 to material in step #6 and mix for 10 minutes.
8. Add Magnesium stearate to the blender and mix for 3 minutes.
9. Transfer the material to Manesty Beta-press and compress the tablets.
10. Coat the tablets with a solution of Opadry II Clear in water using a O'Hara Labcoat.

### Example 3

### Manufacturing Details for Enteric Coating (Both 75 and 225 mg)

After step #9 from the previous example:
11. Coat the tablets with a suspension of Eudragit L in water.
12. Coat the material in step # 11 with Opadry white.

The polymer we will be using for the enteric part will be one of the following:

| | |
|---|---|
| Eudragit L | From Degussa or |
| | Rohm Pharma |
| Eudragit L 50D | From Degussa or |
| | Rohm Pharma |
| Acryl-eze (methacrylic acid co-polymer type C) | From Colorcon |
| Sureteric (polyvinyl acetate phthalate) | From Colorcon |

### Example 4

### Manufacturing Details for Oral Enterically Coated Sustained Release Tablets

Ingredients used:

| | |
|---|---|
| Methylnaltrexone | 250 g |
| Docusate sodium | 100 g |
| Lactose | 20 g |
| Hydroxypropyl methylcellulose (1000 cps) | 120 g |
| Polyvinylpyrrolidone | 10 g |
| Dibasic calcium phosphate | 50 g |
| Magnesium stearate | 3 g |
| Cellulose acetate phthalate | 50 g |
| Water | as needed |

Manufacturing steps:
1. Mix 250 g of methylnaltrexone with the 100 g of docusate sodium in a high shear blender.
2. Add 20 g of lactose and 120 g of hydroxypropyl methylcellulose to the blender and mix thoroughly.
3. Granulate the above mixture using a solution of polyvinylpyrrolidone in water (10 g in 100 ml).
4. After the granules are formed, transfer the material to a fluidized bed drier and dry the mixture.
5. Pass the mixture from step 4 thru a mill to reduce the particle size of the granules to make it more uniform.
6. Add the material from step 5 to a tumble blender and add 50 g of dibasic calcium phosphate and mix thoroughly for 10 minutes.
7. Add 3 g of magnesium stearate to the blender and mix for 3 to 5 minutes.
8. Transfer the material to a tablet press and compress into tablets with a target weight of 553 mg per tablet.
9. Coat the tablets from step 8, in a perforated pan, with cellulose acetate phthalate to a tablet weight of 603 mg.

### Example 5

### Manufacturing Details for a Suppository

Ingredients used:

| | |
|---|---|
| Methylnaltrexone | 250 g |
| Glycerin | 500 g |
| Polyethylene glycol 1000 | 100 g |
| Polyethylene glycol 4000 | 800 g |

Manufacturing steps:
1. In a jacketed pot, add 250 g of methylnaltrexone and 500 g of glycerin and start mixing.
2. Add 100 g of polyethylene glycol 1000 and 800 g of polyethylne glycol 4000 to the materials in step 1 and continue mixing.
3. The material from step 2 is heated via the jacket to render a flowable and pourable mixture.
4. The mixture is poured into containers for manufacturing suppositories and allowed to cool to room temperature.
5. Solidified suppositories are then harvested from the containers. Each suppository would weigh 1650 mg.

1. A method for treating irritable bowel syndrome comprising administering to a patient in need of such treatment an amount of a pharmaceutical preparation comprising a peripheral opioid antagonist effective to ameliorate at least one symptom of the irritable bowel syndrome, wherein the pharmaceutical preparation is free of bioavailable calcium or salts thereof.

2. The method of claim 1 wherein the pharmaceutical preparation is administered parenterally.

3. The method of claim 2 wherein the pharmaceutical preparation is administered from a route selected from the group consisting of intravenously, subcutaneously, via a needleless injection, and via infusion.

4. The method of claim 3 wherein the pharmaceutical preparation is administered intravenously.

5. The method of claim 3 wherein the pharmaceutical preparation is administered subcutaneously.

6. The method of claim 3 wherein the pharmaceutical preparation is administered via a needleless injection.

7. The method of claim 3 wherein the pharmaceutical preparation is administered via an infusion.

8. The method of claim 1 wherein the pharmaceutical preparation is administered intrarectally.

9. The method of claim 1 wherein the pharmaceutical preparation is administered transdermally.

10. The method of claim 1 wherein the pharmaceutical preparation is administered intranasally.

11. The method of claim 1 wherein the pharmaceutical preparation is administered as a solution.

12. The method of claim 1 wherein the pharmaceutical preparation is administered as a suppository.

13. The method of claim 1 wherein the pharmaceutical preparation is administered as an enema.

14. The method of claim 1 wherein the pharmaceutical preparation is administered as a tablet or capsule.

15. The method of claim 1 wherein the patient is not undergoing exogenous opioid treatment.

16. The method of claim 1 wherein the patient is female.

17. The method of claim 1 wherein the patient is male.

18. The method of claim 1 wherein the patient is a child.

19. The method of claim 1 wherein the symptom is diarrhea.

20. The method of claim 1 wherein the symptom is alternating constipation and diarrhea.

21. The method of claim 1 wherein the symptom is constipation.

22. The method of claim 1 wherein the symptom is constipation and abdominal pain.

23. The method of claim 1 wherein the symptom is abdominal bloating.

24. The method of claim 1 wherein the symptom is abdominal distension.

25. The method of claim 1 wherein the symptom is abnormal stool frequency.

26. The method of claim 1 wherein the symptom is abnormal stool consistency.

27. The method of claim 1 wherein the symptom is abdominal pain.

28. The method of claim 1 further comprising administering an antibiotic to the patient.

29. The method of claim 1 further comprising administering an opioid agonist to the patient.

30. The method of claim 1 further comprising administering at least one irritable bowel syndrome therapeutic agent to the patient.

31. The method of claim 30, further comprising administering an opioid agonist to the patient.

32. The method of claim 30, wherein the irritable bowel syndrome therapeutic agent is selected from the group consisting of antispasmodics, anti-muscarinics, antiinflammatory agents, pro-motility agents, 5HT₁ agonists, 5HT₃ antagonists, 5HT₄ antagonists, 5HT₄ agonists, bile salt sequestering agents, bulk-forming agents, alpha2-adrenergic agonists, mineral oils, antidepressants, herbal medicines, and combinations thereof.

33. The method of claim 30, wherein the irritable bowel syndrome agent is not a 5HT₃ antagonist, a 5HT₄ antagonist, or a 5HT₄ agonist.

34. The method of claim 30 wherein the irritable bowel syndrome therapeutic agent is an antidiarrheal medication.

35. The method of claim 30 wherein the irritable bowel syndrome therapeutic agent is an antidepressant.

36. The method of claim 30 wherein the irritable bowel syndrome therapeutic agent is an herbal medicine.

37. The method of claim 30 wherein the irritable bowel syndrome therapeutic agent is an alpha₂-adrenergic agent.

38. The method of claim 30 wherein the agent is a 5HT₄ agonist.

39. The method of claim 38, wherein the 5HT₄ agonist is 3-(5-methoxy-IM-indole-3-yl-methylene)-N-pentylcarbazimidamide.

40. The method of claim 30 wherein the agent is polyethylene glycol 3350.

41. The method of claim 1 wherein the peripheral opioid antagonist is a quaternary derivative of noroxymorphone.

42. The method of claim 41 wherein the quaternary derivative of noroxymorphone is methylnaltrexone.

43. The method of claim 41 wherein the amount of the quaternary derivative of noroxymorphone ranges from 1.0 to 3.0 mg/kg.

44. The method of claim 43 wherein the quaternary derivative of noroxymorphone is methylnaltrexone.

45. The method of claim 41 wherein the amount of the peripheral opioid antagonist ranges from 0.1 to 0.45 mg/kg.

46. The method of claim 42 wherein the amount of the quaternary derivative of noroxymorphone ranges from 0.1 to 0.45 mg/kg.

47. The method of claim 3 wherein the pharmaceutical preparation is administered by infusion.

48. The method of claim 40 wherein the amount of peripheral opioid antagonist is effective to achieve a mean peak plasma concentration of 1400 ng/ml or less of peripheral opioid antagonist.

49. The method of claim 48 wherein the mean peak plasma concentration is 1200 ng/ml or less of peripheral opioid antagonist.

50. The method of claim 48 wherein the mean peak plasma concentration is 1000 ng/ml or less of peripheral opioid antagonist.

51. A method for treating irritable bowel syndrome comprising orally administering to a patient in need of such treatment an amount of a pharmaceutical preparation comprising a peripheral opioid antagonist effective to ameliorate at least one symptom of the irritable bowel syndrome, wherein the pharmaceutical preparation is free of bioavailable calcium or salts thereof.

52. The method of any one of claim 51 wherein the pharmaceutical preparation is administered in an enteric coated formulation.

53. The method of any one of claim 51 wherein the pharmaceutical preparation is administered in a sustained release formulation.

54. The method of any one of claim 51 wherein the pharmaceutical preparation is administered in an enteric coated sustained release formulation.

55. The method of any of one claim 51 wherein the pharmaceutical preparation is administered in a colonic site-directed formulation.

56. The method of claim 51 wherein the patient is not undergoing exogenous opioid treatment.

57. The method of claim 51 wherein the patient is female.

58. The method of claim 51 wherein the patient is male.

59. The method of claim 51 wherein the patient is a child.

60. The method of claim 51 wherein the symptom is constipation.

61. The method of claim 51 wherein the symptom is constipation and abdominal pain.

62. The method of claim 51 wherein the symptom is diarrhea.

63. The method of claim 51 wherein the symptom is alternating constipation and diarrhea.

64. The method of claim 51 wherein the symptom is abdominal bloating.

65. The method of claim 51 wherein the symptom is abdominal distension.

66. The method of claim 51 wherein the symptom is abnormal stool frequency.

67. The method of claim 51 wherein the symptom is abnormal stool consistency.

68. The method of claim 51 wherein the symptom is abdominal pain.

69. The method of claim 51 further comprising administering an antibiotic to the patient.

70. The method of claim 51 further comprising administering at least one irritable bowel syndrome therapeutic agent.

71. The method of claim 70 wherein the irritable bowel syndrome therapeutic agent is an antidepressant.

72. The method of claim 70 wherein the irritable bowel syndrome therapeutic agent is an antidiarrheal medication.

73. The method of claim 70 wherein the irritable bowel syndrome therapeutic agent is an herbal medicine.

74. The method of claim 70 wherein the irritable bowel syndrome therapeutic agent is an opioid agonist.

75. The method of claim 70 wherein the irritable bowel syndrome therapeutic agent is an alpha₂-adrenergic agent.

76. The method of claim 70 wherein the irritable bowel syndrome therapeutic agent is a 5-HT₄ agonist.

77. The method of claim 65 wherein the 5-HT₄ agonist is 3-(5-methoxy-IM-indole-3-yl-methylene)-N-pentylcarbazimidamide.

78. The method of claim 70 wherein the irritable bowel syndrome therapeutic agent is not a 5-HT₃ antagonist, a 5-HT₄ antagonist or a 5-HT₄ agonist.

79. The method of claim 76 wherein the irritable bowel syndrome therapeutic agent is a polyethylene glycol 3350.

80. The method of claim 51 wherein the peripheral opioid antagonist is a quaternary derivative of noroxymorphone.

81. The method of claim 80 wherein the quaternary derivative of noroxymorphone is methylnaltrexone.

82. The method of claim 81 wherein the amount ranges from 50 to 750 mg/day.

83. The method of claim 81 wherein the amount is 75 mg of the quaternary derivative of noroxymorphone.

84. The method of claim 81 wherein the amount is 225 mg of the quaternary derivative of noroxymorphone.

85. A pharmaceutical preparation comprising a quaternary derivative of noroxymorphone and an irritable bowel syndrome therapeutic agent and a pharmaceutically acceptable carrier.

86. The pharmaceutical preparation of claim 85 wherein the quaternary derivative of noroxymorphone is methylnaltrexone.

87. The pharmaceutical preparation of claim 85 or 86 wherein the pharmaceutical preparation is free of bioavailable calcium or salts thereof.

88. The pharmaceutical preparation of claim 85 wherein the irritable bowel syndrome therapeutic agent is selected from the group consisting of antispasmodics, anti-muscarinics, antiinflammatory agents, pro-motility agents, 5HT₁ agonists, 5HT₃ antagonists, 5HT₄ antagonists, 5HT₄ agonists, bile salt sequestering agents, bulk-forming agents, alpha₂-adrenergic agonists, mineral oils, antidepressants, herbal medicines and combinations thereof.

89. The pharmaceutical preparation of claim 85 wherein the irritable bowel syndrome therapeutic agent is an antispasmodic.

90. The pharmaceutical preparation of claim 85 wherein the irritable bowel syndrome therapeutic agent is an anti-muscarinic.

91. The pharmaceutical preparation of claim 85 wherein the irritable bowel syndrome therapeutic agent is an antiinflammatory agent.

92. The pharmaceutical preparation of claim 85 wherein the irritable bowel syndrome therapeutic agent is a pro-motility agent.

93. The pharmaceutical preparation of claim 85 wherein the irritable bowel syndrome therapeutic agent is a 5HT₁ agonist, a 5HT₃ antagonist or a 5HT₄ agonist.

94. The pharmaceutical preparation of claim 85 wherein the irritable bowel syndrome therapeutic agent is not a 5HT₃ antagonist, a 5HT₄ antagonist or a 5HT₄ agonist.

95. The pharmaceutical preparation of claim 85 wherein the irritable bowel syndrome therapeutic agent is a 5HT₄ agonist.

96. The pharmaceutical preparation of claim 95 wherein the irritable bowel syndrome therapeutic agent is 3-(5-methoxy-IM-indole-3-yl-methylene)-N-pentylcarbazimidamide.

97. The pharmaceutical preparation of claim 85 wherein the irritable bowel syndrome therapeutic agent is a bile salt sequestering agent.

98. The pharmaceutical preparation of claim 85 wherein the irritable bowel syndrome therapeutic agent is a bulk-forming agent.

99. The pharmaceutical preparation of claim 85 wherein the irritable bowel syndrome therapeutic agent is an alpha2-adrenergic agonist.

100. The pharmaceutical preparation of claim 85 wherein the irritable bowel syndrome therapeutic agent is a mineral oil.

101. The pharmaceutical preparation of claim 85 wherein the irritable bowel syndrome therapeutic agent is an antidepressant.

102. The pharmaceutical preparation of claim 85 wherein the irritable bowel syndrome therapeutic agent is an herbal medicine.

103. The pharmaceutical preparation of claim 85 wherein the pharmaceutical preparation is formulated for oral administration.

104. The pharmaceutical preparation of claim 102 wherein the formulation is selected from the group consisting of a capsule, a powder, a granule, a crystal, a tablet, a solution, an extract, a suspension, a soup, a syrup, an elixir, a tea, a liquid-filled capsule, an oil, a chewable tablet, a chewable piece, an enteric-coated tablet, a sustained release tablet or capsule, and an enteric-coated sustained release tablet.

105. The pharmaceutical preparation of claim 85 wherein the pharmaceutical preparation is formulated for rectal administration.

106. The pharmaceutical preparation of claim 105 wherein the formulation is selected from the group consisting of a suspension, a solution, a suppository, an oil, and an enema.

107. The pharmaceutical preparation of claim 85 wherein the pharmaceutical preparation is formulated for a route of administration selected from the group consisting of sublingual, intranasal, transdermal, intradermal, intramuscular, subcutaneous, injectable, and infusion.

108. A kit comprising:
a package containing a peripheral opioid antagonist preparation, wherein the preparation is free of bioavailable calcium and salts thereof; and
instructions for using the preparation to treat irritable bowel syndrome.

109. The kit of claim 108, further comprising an antibiotic.

110. The kit of claim 108, further comprising an irritable bowel syndrome therapeutic agent

111. The kit of claim 108, wherein the preparation is a pharmaceutical preparation according to claim 85.
